(19) United States
(12) Patent Application Publication
(10) Pub. No.: US 2026/0182914 A1
(43) Pub. Date: Jul. 2, 2026

(54) BRAID FABRIC, WEARABLE PHYSIOLOGICAL SIGNAL DETECTION DEVICE USING THE SAME AND PHYSIOLOGICAL SIGNAL CORRECTION METHOD

(71) Applicant: INDUSTRIAL TECHNOLOGY RESEARCH INSTITUTE, Chutung (TW)

(72) Inventors: Ming-Ya HUNG, Taoyuan City (TW); Jo-Ping LEE, Hsinchu City (TW); Yu-Yuan CHEN, Zhubei City (TW); Po-Yen LIU, Taoyuan City (TW); Chun-Yu CHAN, Hsinchu City (TW); Jun-Chao ZHAO, Tainan City (TW)

(73) Assignee: INDUSTRIAL TECHNOLOGY RESEARCH INSTITUTE, Chutung (TW)

(21) Appl. No.: 19/005,170

(22) Filed: Dec. 30, 2024

(30) Foreign Application Priority Data
Dec. 26, 2024 (TW) 113150914

(51) Int. Cl. A61B 5/00

(52) U.S. Cl. CPC A61B 5/6804; A61B 5/7225; A61B 2562/0261

(57) ABSTRACT

A wearable physiological signal detection device includes a braid fabric, a plurality of electrodes, a physiological signal analysis unit and a strain signal analysis unit. The braid fabric includes an elastic fiber layer and a strain-sensing fiber layer. The strain-sensing fiber layer is assembled together with the elastic fiber layer. The electrodes are disposed on the braid fabric. The physiological signal analysis unit is electrically connected to the electrodes and configured to obtain a physiological signal from the electrodes. The strain signal analysis unit is electrically connected to the physiological signal analysis unit and the strain-sensing fiber layer and configured to obtain a gain value according to a strain-sensing signal sensed by the strain-sensing fiber layer and correct the physiological signal according to the gain value.

This application claims the benefit of Taiwan application Serial No. 113150914, filed Dec. 26, 2024, the subject matter of which is incorporated herein by reference.

### TECHNICAL FIELD

The technical field relates to a braid fabric, a wearable physiological signal detection device using the same and a physiological signal correction method using the same.

### BACKGROUND

When a patient uses a wearable physiological signal detection device, the wearable physiological signal detection device will be attached to the skin surface to detect the physiological state of the human body. However, the muscle movement of the human body may cause changes in the tightness of the wearable physiological signal detection device, and it will change the distance between the electrodes of the wearable physiological signal detection device and the skin, resulting in impedance fluctuations between the electrodes and the skin, thereby may affect the signal quality and even lead to severe distortion of physiological signals. Therefore, how to submit a technology that may improve the aforementioned problems is one of the goals of those in this technical field.

### SUMMARY

According to an embodiment, a braid fabric is provided. The braid fabric includes an elastic fiber layer and a strain-sensing fiber layer. The strain-sensing fiber layer is assembled together with the elastic fiber layer.

According to another embodiment, a wearable physiological signal detection device is provided. The wearable physiological signal detection device includes a braid fabric, a plurality of electrodes, a physiological signal analysis unit and a strain signal analysis unit. The braid fabric includes an elastic fiber layer and a strain-sensing fiber layer. The strain-sensing fiber layer is assembled together with the elastic fiber layer. The electrodes are disposed on the braid fabric. The physiological signal analysis unit is electrically connected to the electrodes and configured to obtain a physiological signal from the electrodes. The strain signal analysis unit is electrically connected to the physiological signal analysis unit and the strain-sensing fiber layer and configured to obtain a gain value according to a strain-sensing signal sensed by the strain-sensing fiber layer and correct the physiological signal according to the gain value.

According to another embodiment, a physiological signal correction method is provided. The physiological signal correction method includes the following steps: obtaining a physiological signal from a plurality of electrodes by a physiological signal analysis unit, wherein the physiological signal analysis unit is electrically connected to the electrodes; obtaining a gain value according to a strain-sensing signal sensed by a strain-sensing fiber layer, by a strain signal analysis unit, wherein the strain signal analysis unit electrically connected to the physiological signal analysis unit and the strain-sensing fiber layer; and correcting the physiological signal according to the gain value by the strain signal analysis unit.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically illustrated in order to simplify the drawing.

### BRIEF DESCRIPTION OF THE DRAWINGS

FIGS. 1A to 1I illustrate the schematic diagrams of configurations of the braid fabrics of a plurality of embodiments of the present disclosure;

FIGS. 2A to 2E illustrate schematic diagrams of cross-sectional views of the braid fabrics along a third direction Z in other embodiments;

FIG. 3 illustrates an expanded schematic diagram of a wearable physiological signal detection device according to an embodiment of the present disclosure;

FIG. 4 illustrates a usage schematic diagram of the wearable physiological signal detection device in FIG. 3;

FIG. 5 illustrates a functional block diagram of the wearable physiological signal detection device in FIG. 3;

FIG. 6A illustrates a schematic diagram of a relationship between the curvature radius of the braid fabric and the strain-sensing signal;

FIG. 6B illustrates a schematic diagram of a relationship between the tightness of the braid fabric and a signal-to-noise ratio;

FIG. 6C illustrates a schematic diagram of a relationship between the tightness of the braid fabric and a root mean square value.

FIG. 7 illustrates a flow chart of a physiological signal correction method of the wearable physiological signal detection device in FIG. 5; and

FIG. 8 illustrates a flow chart of the physiological signal correction method of the wearable physiological signal detection device in FIG. 5 according to another embodiment.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

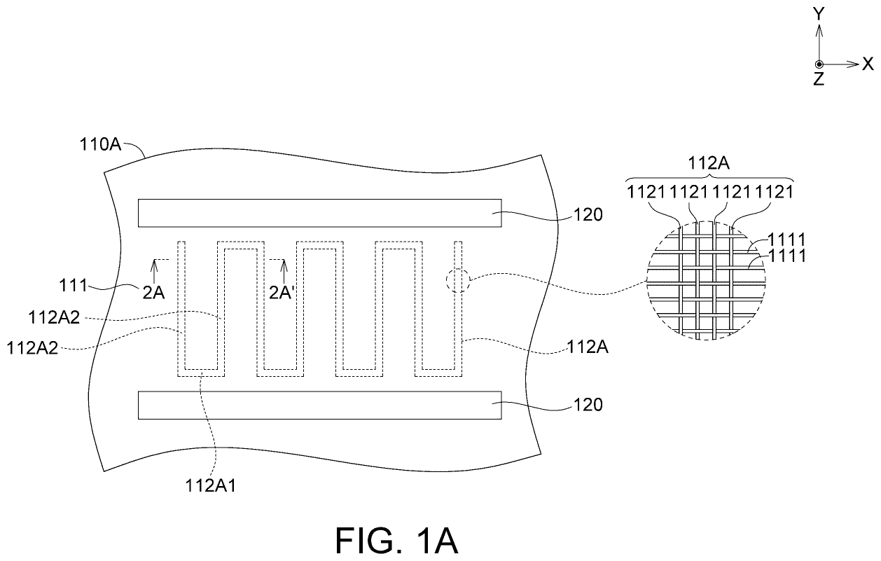

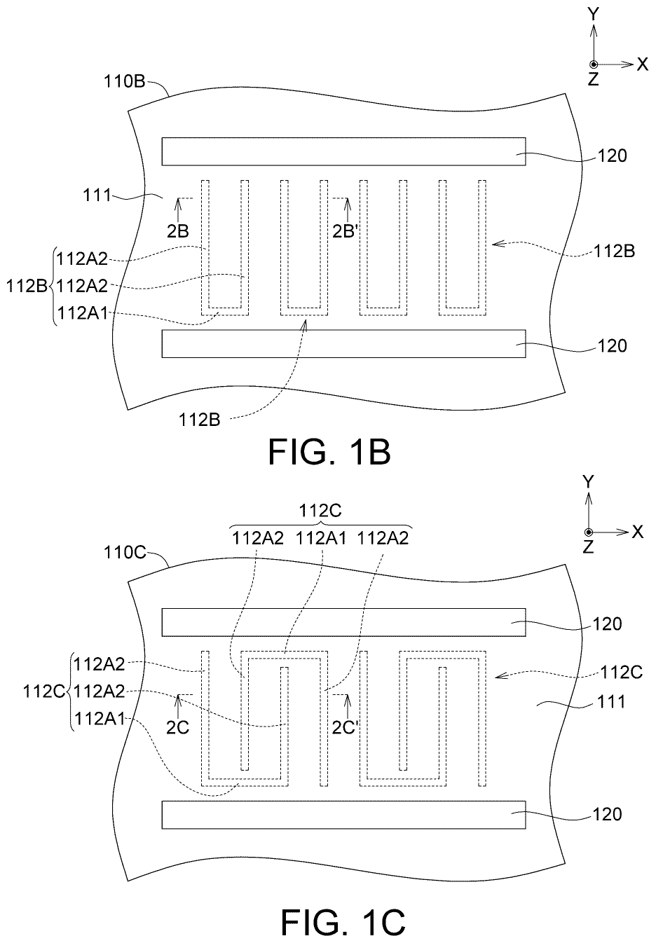

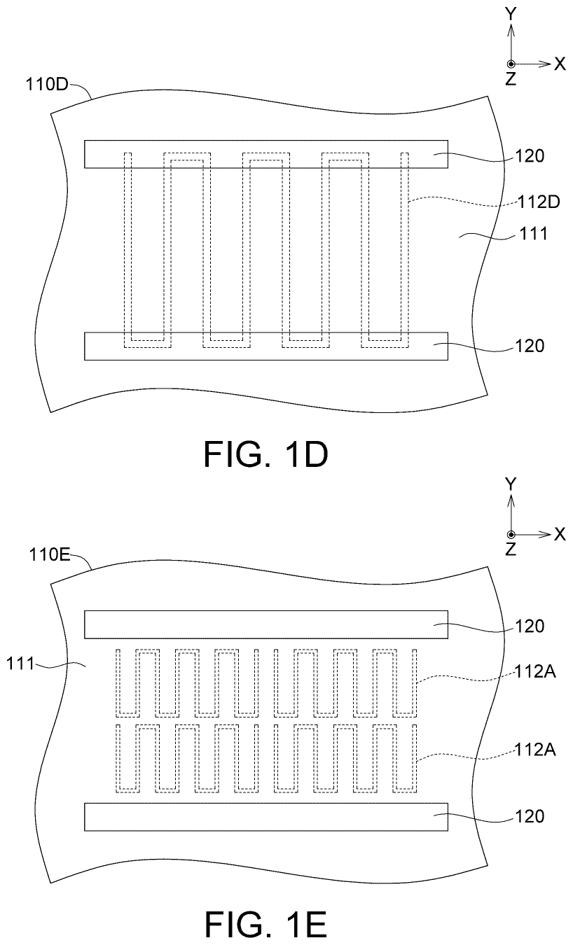

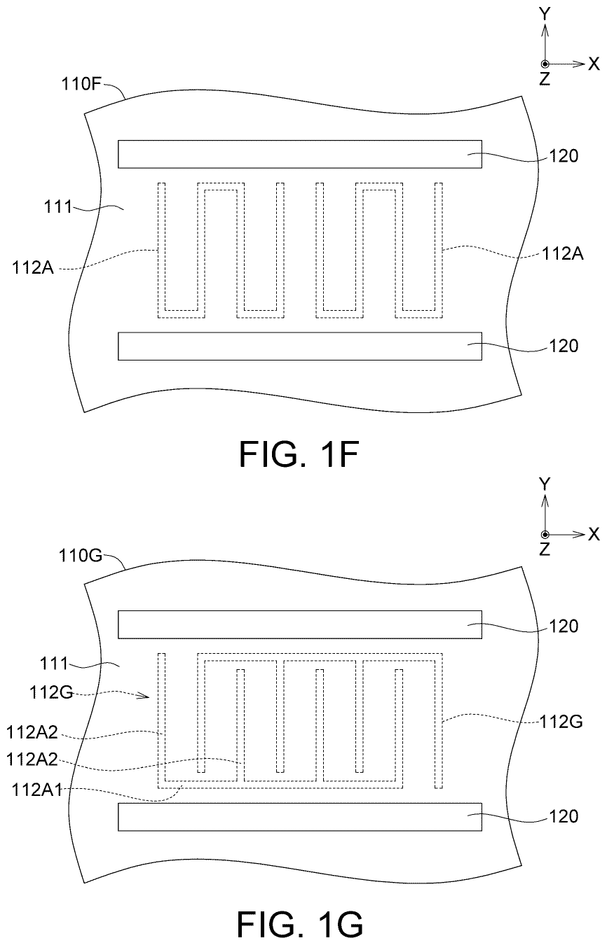

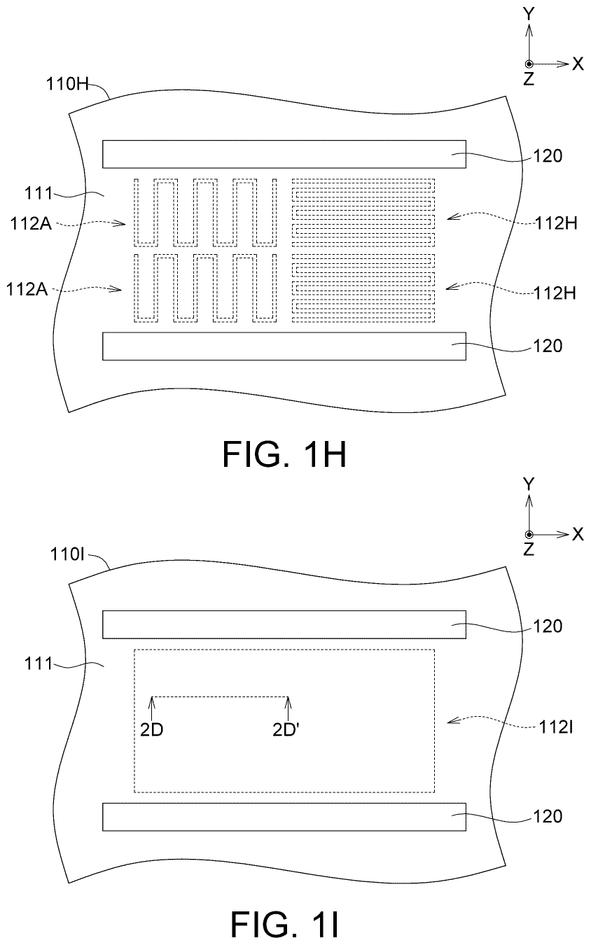

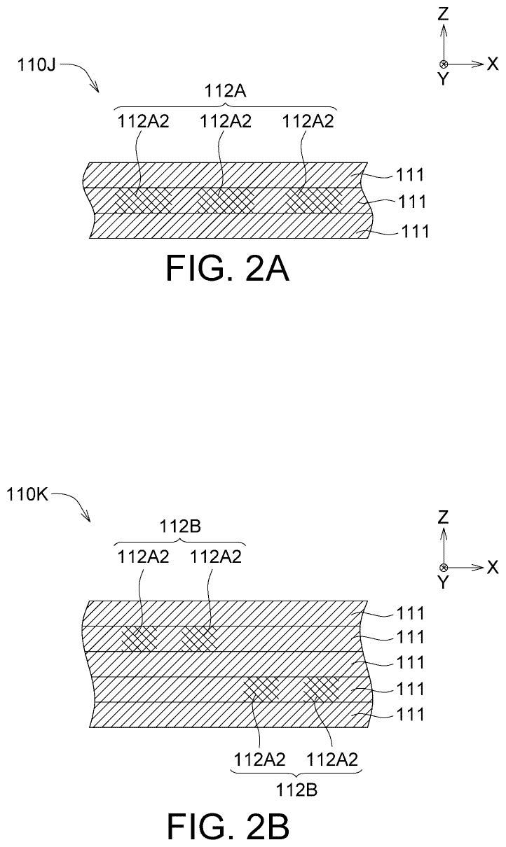

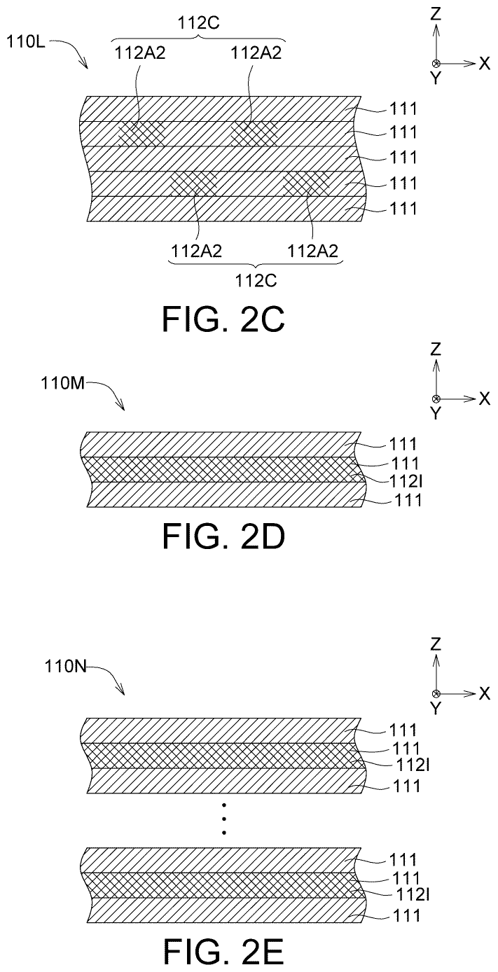

Referring to FIGS. 1A to 1I and 2A to 2E, FIGS. 1A to 1I illustrate the schematic diagrams of configurations of the braid fabrics 110A to 110I of a plurality of embodiments of the present disclosure, and FIGS. 2A to 2E illustrate schematic diagrams of cross-sectional views of the braid fabrics 110J to 110N along a third direction Z in other embodiments.

As illustrated in FIG. 1A, the braid fabric 110A includes at least one elastic fiber layer 111 and at least one strain-sensing fiber layer 112A. The strain-sensing fiber layer 112A and the elastic fiber layer 111 are assembled together. In an embodiment, the elastic fiber layer 111 includes a plurality of elastic fibers 1111, and the strain-sensing fiber layer 112A includes a plurality of strain-sensing fibers 1121. The elastic fibers 1111 and the strain-sensing fibers 1121 are intertwined with each other. Although the strain-sensing fibers 1121 illustrated in FIG. 1A are separated from each other, in fact, the strain-sensing fibers 1121 may be the same one fiber interwoven back and forth among the elastic fibers 1111. The elastic fiber layer 111 of the braid fabric 110A and the strain-sensing fiber layer 112A are woven together and finally form an integrated fabric structure. The strain-sensing fiber layer is used to provide immediate and continuous feedback on the wearing deformation condition and generate a strain-sensing signal (described later) for correcting a physiological signal.

As illustrated in FIG. 1A, the strain-sensing fiber layer 112A may be disposed between two electrodes 120 (described later). In terms of the extension method of the strain-sensing fiber layer, the strain-sensing fiber layer 112A includes a plurality of first extending portions 112A1 and a plurality of second extending portions 112A2. The first extending portions 112A1 may extend along a first direction X, and the second extending portions 112A2 may extend along a second direction Y, wherein the first direction X and the second direction Y are substantially perpendicular. In another embodiment, the first extending portion 112A1 and the second extending portion 112A2 may respectively extend along two non-perpendicular directions. Each first extending portion 112A1 may connect two adjacent second extending portions 112A2. The strain-sensing fiber layer 112A may extend continuously along a circuitous path.

As illustrated in FIG. 1B, the braid fabric 110B includes an elastic fiber layer 111 and a plurality of strain-sensing fiber layers 112B, wherein the elastic fiber layer 111 and the strain-sensing fiber layer 112B are assembled together. The braid structure of the strain-sensing fiber layer 112B is the same as or similar to the braided structure of the strain-sensing fiber layer 112A in FIG. 1A, and it will not be repeated again. The braid fabric 110B has the structures the same as or similar to that of the braid fabric 110A, and at least one difference is that the braid fabric 110B includes a plurality of strain-sensing fiber layers 112B which are separately disposed, wherein each strain-sensing fiber layer 112B includes a first extending portion 112A1 and two second extending portions 112A2, wherein the first extending portion 112A1 may be connected to the two second extending portions 112A2.

As illustrated in FIG. 1C, the braid fabric 110C includes the elastic fiber layer 111 and a plurality of strain-sensing fiber layers 112C, wherein the elastic fiber layer 111 and the strain-sensing fiber layer 112C are assembled together. These strain-sensing fiber layers 112C are disposed separately from each other. The braided structure of the strain-sensing fiber layer 112C is the same as or similar to the braided structure of the strain-sensing fiber layer 112A in FIG. 1A, and it will not be repeated again. Each strain-sensing fiber layer 112C includes the first extending portion 112A1 and two second extending portions 112A2, wherein the first extending portion 112A1 may connect the two second extending portions 112A2. These strain-sensing fiber layers 112C have the structure same as or similar to that of the aforementioned strain-sensing fiber layer 112B, and at least one difference is that the second extending portion 112A2 of one of the two strain-sensing fiber layers 112C may be located between two second extending portions 112A2 of another of the two strain-sensing fiber layers 112C.

As illustrated in FIG. 1D, the braid fabric 110D includes the elastic fiber layer 111 and a strain-sensing fiber layer 112D, wherein the elastic fiber layer 111 and the strain-sensing fiber layer 112D are assembled together. The braided structure of the strain-sensing fiber layer 112D is the same as or similar to the braided structure of the strain-sensing fiber layer 112A in FIG. 1A, and it will not be repeated again. The extension manner of the strain-sensing fiber layer 112D may be similar to that of the aforementioned strain-sensing fiber layer 112A in FIG. 1A, and at least one difference is that the strain-sensing fiber layer 112D partially overlaps the electrode 120 along the third direction Z.

As illustrated in FIG. 1E, the braid fabric 110E includes the elastic fiber layer 111 and a plurality of strain-sensing fiber layers 112A, wherein the elastic fiber layer 111 and the strain-sensing fiber layer 112A are assembled together. The strain-sensing fiber layers 112A are separated from each other. The braid fabric 110E has the structure same as or similar to that of the aforementioned braid fabric 110A, and at least one difference is that a plurality of the strain-sensing fiber layers 112A may be disposed between the two electrodes 120 and may be disposed along the first direction X and the second direction Y.

As illustrated in FIG. 1F, the braid fabric 110F includes the elastic fiber layer 111 and a plurality of the strain-sensing fiber layers 112A, wherein the elastic fiber layer 111 and the strain-sensing fiber layer 112A are assembled together. The strain-sensing fiber layers 112A are separated from each other. The braid fabric 110F has the structure same as or similar to that of the braid fabric 110A, and at least one difference is that a plurality of the strain-sensing fiber layers 112A of the braid fabric 110F may be disposed between the two electrodes 120 and may be disposed along the first direction X.

As illustrated in FIG. 1G, the braid fabric 110G includes the elastic fiber layer 111 and a plurality of strain-sensing fiber layers 112G, wherein the elastic fiber layer 111 and the strain-sensing fiber layer 112G are assembled together. These strain-sensing fiber layers 112G may be disposed separately. The braided structure of the strain-sensing fiber layer 112G is the same as or similar to the braided structure of the strain-sensing fiber layer 112A in FIG. 1A, and it will not be repeated again. Each strain-sensing fiber layer 112G includes the first extending portion 112A1 and a plurality of the second extending portions 112A2, wherein the second extending portions 112A2 are connected to the first extending portion 112A1. The strain-sensing fiber layer 112G has the structure same as or similar to that of the aforementioned strain-sensing fiber layer 112C, and at least one difference is that more second extending portions 112A2 are connected to the first extending portions 112A1. In addition, at least one second extending portion 112A2 of one of the two strain-sensing fiber layers 112G may be located between two second extending portions 112A2 of the other of the two strain-sensing fiber layers 112G.

As illustrated in FIG. 1H, the braid fabric 110H includes the elastic fiber layer 111, a plurality of the first strain-sensing fiber layers 112A and a plurality of second strain-sensing fiber layers 112H, wherein the elastic fiber layer 111, the first strain-sensing fiber layers 112A and the second strain-sensing fiber layers 112H are assembled together. A plurality of the first strain-sensing fiber layers 112A and a plurality of the second strain-sensing fiber layers 112H may be disposed between the two electrodes 120. The extension manner of the second strain-sensing fiber layers 112H may be similar to that of the aforementioned strain-sensing fiber layers 112A; however, the second strain-sensing fiber layer 112H and the first strain-sensing fiber layer 112A are arranged in different orientations, for example, the arrangement orientations of the first strain-sensing fiber layer 112A and the second strain-sensing fiber layer 112H may differ by 90 degrees.

As illustrated in FIG. 1I, the braid fabric 110I includes the elastic fiber layer 111 and a strain-sensing fiber layer 112I, wherein the elastic fiber layer 111 and the strain-sensing fiber layer 112I are assembled together. The braid fabric 110I has the structures same as or similar to the aforementioned braid fabric, and at least one difference is that the strain-sensing fiber layer 112I of the braid fabric 110I is spread between the two electrodes 120 without a specific pattern. In other words, the strain-sensing fiber layer 112I is a strain-sensing fiber layer without patterning (that is, an intact fiber layer).

In summary, it may be seen that the embodiments of the present disclosure do not limit the extension manner of the strain-sensing fiber layer. The strain-sensing fiber layer may extend along a straight line, a curve, and combinations thereof. In addition, the entire or all strain-sensing fiber layer may be disposed between the two electrodes, or the strain-sensing fiber layer may partially extend to at least one of the two electrodes. In addition, a plurality of the strain-sensing fiber layers may be arranged side by side along the first direction X and/or the second direction Y or arranged along an axial direction intersecting the first direction X (or the second direction Y). In addition, two of the strain-sensing fiber layers may have the same or different extension patterns.

A strain-sensing fiber layer of the braid fabric may be located in the same layer of the braid fabric or may extend to a plurality of layers of different heights in the braid fabric. Alternatively, multiple a plurality of the strain-sensing fiber layers of the braid fabric may be disposed in the same layer or in a plurality of layers at different heights. The following introduce the cross-sectional structures of braid fabrics in various embodiments with using FIGS. 2A to 2E.

As illustrated in FIG. 2A, the cross-sectional structure of the strain-sensing fiber layer 112A is, for example, the cross-section of the strain-sensing fiber layer 112A along a direction 2A-2A′ in FIG. 1A. The braid fabric 110J may include a plurality of the elastic fiber layers 111 and the strain-sensing fiber layer 112A, wherein the strain-sensing fiber layer 112A is disposed on one of the elastic fiber layers 111, for example, a middle one of the three elastic fiber layers 111. The strain-sensing fiber layer 112A is located between two outermost elastic fiber layers 111, that is, the strain-sensing fiber layer 112A is covered by the two outermost elastic fiber layers 111. In addition, the strain-sensing fiber layer 112A in FIG. 2A may also be replaced by the strain-sensing fiber layer in FIGS. 1B to 1H.

As illustrated in FIG. 2B, the cross-sectional structure of a plurality of the strain-sensing fiber layers 112B is, for example, the cross-section of a plurality of the strain-sensing fiber layers 112B along a direction 2B-2B′ in FIG. 1B. The braid fabric 110K includes the cross-sectional structure same as or similar to that of the aforementioned braid fabric 110J, and at least one difference is that the braid fabric 110K may include a plurality of the elastic fiber layers 111 and a plurality of the strain-sensing fiber layers 112B, wherein each strain-sensing fiber layer 112B may be disposed in one of the elastic fiber layers 111, for example, in the middle one of three consecutive elastic fiber layers 111. In the present embodiment, a plurality of the strain-sensing fiber layers 112B are respectively disposed in a plurality of the elastic fiber layers 111 at different heights. The strain-sensing fiber layers 112B are located between the two outermost elastic fiber layers 111, that is, the strain-sensing fiber layers 112B are covered by the two outermost elastic fiber layers 111. In addition, the strain-sensing fiber layer 112B in FIG. 2B may also be replaced by the strain-sensing fiber layer in FIGS. 1B to 1H.

As illustrated in FIG. 2C, the cross-sectional structure of a plurality of the strain-sensing fiber layers 112C is, for example, the cross-section of a plurality of the strain-sensing fiber layers 112C along a direction 2C-2C′ in FIG. 1C. The braid fabric 110L may include a plurality of the elastic fiber layers 111 and a plurality of the strain-sensing fiber layers 112C, wherein each strain-sensing fiber layer 112C may be disposed in one of the elastic fiber layers 111, for example, disposed on the middle one of the three consecutive ones of the elastic fiber layers 111. In the present embodiment, a plurality of the strain-sensing fiber layers 112C are respectively disposed in a plurality of the elastic fiber layers 111 at different heights. These strain-sensing fiber layers 112C are located between the two outermost elastic fiber layers 111, that is, these strain-sensing fiber layers 112C are covered by the two outermost elastic fiber layers 111. In addition, the strain-sensing fiber layer 112C in FIG. 2C may also be replaced by the strain-sensing fiber layer in FIGS. 1B to 1H.

As illustrated in FIG. 2D, the cross-sectional structure of the strain-sensing fiber layer 112I is, for example, the cross-section of the strain-sensing fiber layer 112I along a direction 2D-2D′ in FIG. 1I. The braid fabric 110M may include a plurality of the elastic fiber layers 111 and a strain-sensing fiber layer 112I. The strain-sensing fiber layer 112I is disposed in one of the elastic fiber layers 111, such as the middle one of the three elastic fiber layers 111.

As illustrated in FIG. 2E, the braid fabric 110N may include a plurality of the elastic fiber layers 111 and a plurality of the strain-sensing fiber layers 112I. Each strain-sensing fiber layer 112I is disposed on one of the elastic fiber layers 111, for example, the middle one of the elastic fiber layers 111, for example, the middle one of three elastic fiber layers 111. The braid fabric 110N has the structures same as or similar to that of the aforementioned braid fabric 110M, and at least one difference is that the braid fabric 110N includes more strain-sensing fiber layers 112I and more elastic fiber layers 111.

In summary, based on the cross-sectional structure of the braid fabric, the braid fabric may include a plurality of the elastic fiber layers and at least one strain-sensing fiber layer. In an embodiment, the strain-sensing fiber layer may be located in one of the elastic fiber layers or extended through or in a plurality of the elastic fiber layers. In another embodiment, a plurality of the strain-sensing fiber layers may be located in the same layer of the elastic fiber layers, or a plurality of the strain-sensing fiber layers may be respectively disposed in the plurality of elastic fiber layers at different heights. In addition, the embodiments of the present disclosure do not limit the number of strain-sensing fiber layers of the braid fabric, which may be determined according to actual needs. In addition, multiple strain-sensing fiber layers located at different height layers may at least partially overlap along the third direction Z or may not overlap at all. In addition, the number of strain-sensing fiber layers located on the same layer may be one or more.

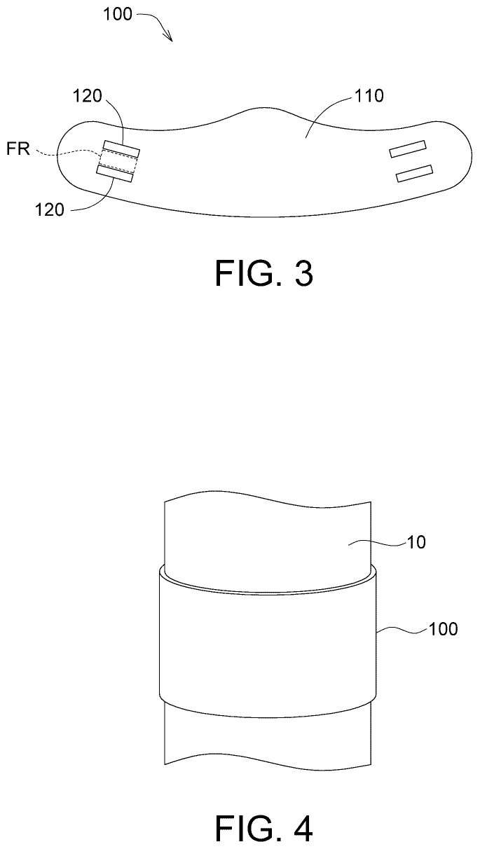

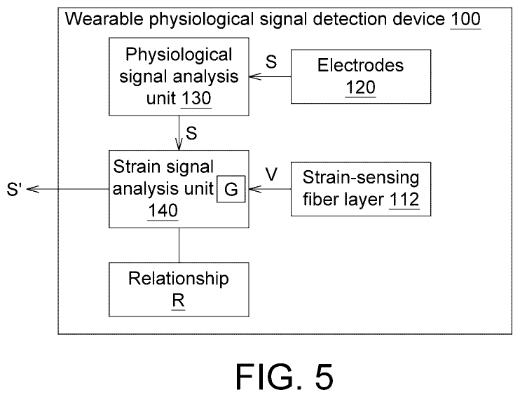

Referring to FIGS. 3 to 5, FIG. 3 illustrates an expanded schematic diagram of a wearable physiological signal detection device 100 according to an embodiment of the present disclosure, FIG. 4 illustrates a usage schematic diagram of the wearable physiological signal detection device 100 in FIG. 3, and FIG. 5 illustrates a functional block diagram of the wearable physiological signal detection device 100 in FIG. 3.

As illustrated in FIGS. 3 and 4, the wearable physiological signal detection device 100 is, for example, knee pad, sock, clothing, pant, neck girth, glove, etc. The wearable physiological signal detection device 100 may be worn on a part of the human body 10, such as knee, palm, ankle, wrist, neck, waist, etc.

As illustrated in FIGS. 3 to 5, the wearable physiological signal detection device 100 includes a braid fabric 110, a plurality of electrodes 120, a physiological signal analysis unit 130 and a strain signal analysis unit 140. The braid fabric 110 illustrated in FIG. 3 has the structure same as or similar to that of at least one of the aforementioned braid fabrics 110A to 110N, and it will not be repeated again here. In an embodiment, the physiological signal analysis unit 130 and the strain signal analysis unit 140 are, for example, physical circuits, such as semiconductor wafers, semiconductor packages, etc. which are formed by using semiconductor processes. In another embodiment, the wearable physiological signal detection device 100 may further include a controller electrically connected to the physiological signal analysis unit 130 and the strain signal analysis unit 140 to control the operations of these units, wherein the controller is, for example, a physical circuit, such as semiconductor wafers, semiconductor packages, etc. which is formed by using semiconductor processes.

As illustrated in FIG. 5, these electrodes 120 are disposed on the braid fabric 110, and these electrodes 120 may detect the physiological signals of the human body 10. The physiological signal analysis unit 130 is electrically connected to the electrodes 120 and is configured to obtain the physiological signal S from the electrodes 120. The strain signal analysis unit 140 is electrically connected to the physiological signal analysis unit 130 and the strain-sensing fiber layer 112 (for example, one of the aforementioned strain-sensing fiber layers 112A, 112B, 112C, 112D, 112H, 112G, 112H and 112I) and configured to: obtain a gain value G according to a strain-sensing signal V sensed by the strain-sensing fiber layer 112; and correct the physiological signal S according to the gain value G. As a result, the wearable physiological signal detection device 100 may automatically detect the tightness of the braid fabric 110 when the physiological signal detection device 100 is worn on the human body 10 and correct the physiological signal S according to the tightness. As a result, the corrected physiological signal S′ corrected by the wearable physiological signal detection device 100 approximates to the actual physiological condition of the human body 10. In addition, the wearable physiological signal detection device 100 may output the corrected physiological signal S′ and display it on a display (not illustrated). For example, the display may be disposed inside or outside the wearable physiological signal detection device 100.

The physiological signal S in this description is, for example, an electromyogram signal which is a fluctuating voltage. The waveform of the fluctuating voltage may depend on the tightness of the braid fabric 110 when the physiological signal detection device 100 is worn on the human body 10, the physiological state of the human body 10 or other factors.

As illustrated in FIG. 5, the strain signal analysis unit 140 may obtain the gain value G corresponding to the strain-sensing signal V according to a relationship R between the strain signal and the gain value. The relationship R between the strain signal and the gain value is, for example, a table, equation, etc. The relationship R between the strain signal and the gain value may be obtained in advance through experiments or simulations and then stored in a memory (not illustrated). Such memory may be disposed in the physiological signal analysis unit 130, the strain signal analysis unit 140 or the aforementioned controller. Alternatively, such memory may be disposed outside the physiological signal analysis unit 130, the strain signal analysis unit 140 or the aforementioned controller, but may be accessed by these components.

As illustrated in Table 1 below, different tightness corresponds to different strain-sensing signals V and different gain values G. The less the numeral of the tightness T is, the tighter the braid fabric 110 on the human body 10 is; otherwise, the looser the braid fabric 110 on the human body 10 is. In addition, the strain-sensing signal V is, for example, a voltage value. The tighter the tightness, the greater the value of the strain-sensing signal V (for example, V1> V2>V3>V4>V5>V6>V7>V8). The gain value G is, for example, any suitable real number. In an embodiment, the looser the tightness, the greater the distortion of the physiological signal S, and the smaller the strain-sensing signal V, so the larger the gain value G is required to compensate for the distorted physiological signal S.

TABLE 1

| tightness T | strain-sensing signal V | gain value G |
|---|---|---|
| 1 | V1 | G1 |
| 2 | V2 | G2 |
| 3 | V3 | G3 |
| 4 | V4 | G4 |
| 5 | V5 | G5 |
| 6 | V6 | G6 |
| 7 | V7 | G7 |
| 8 | V8 | G8 |

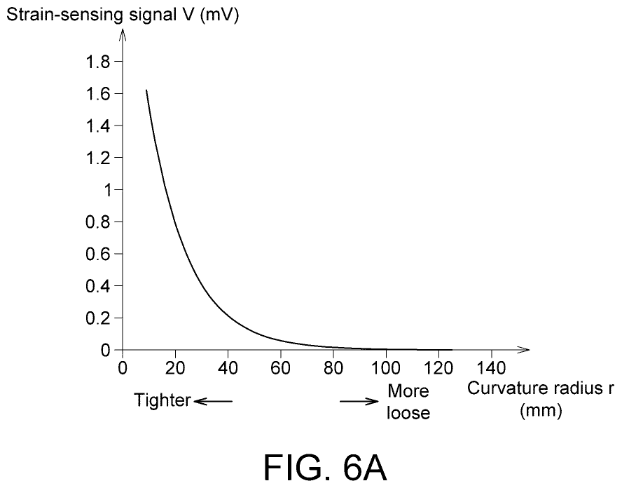

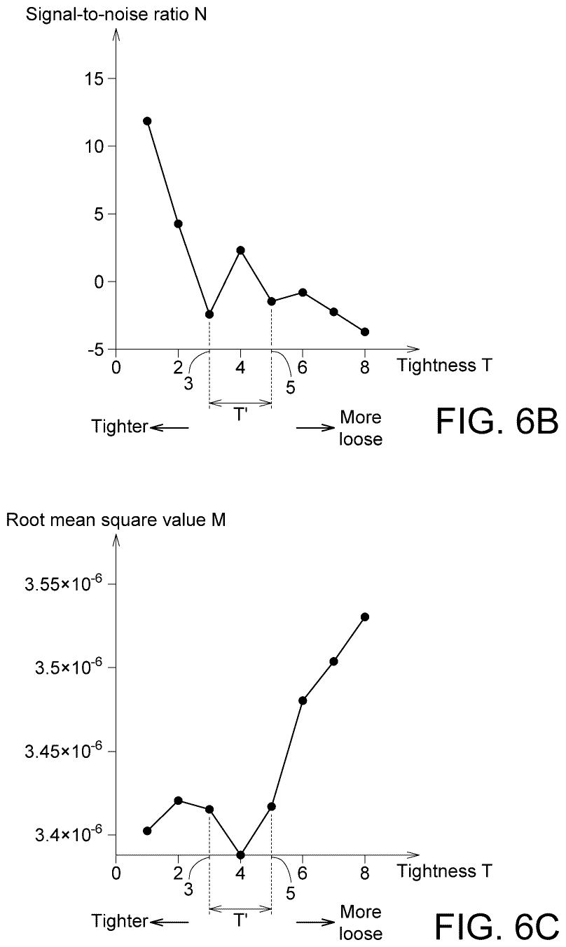

Referring to FIGS. 6A to 6C, FIG. 6A illustrates a schematic diagram of a relationship between the curvature radius r of the braid fabric 110 and the strain-sensing signal V, FIG. 6B illustrates a schematic diagram of a relationship between the tightness of the braid fabric 110 and a signal-to-noise ratio N, and FIG. 6C illustrates a schematic diagram of a relationship between the tightness of the braid fabric 110 and a root mean square value M.

The strain signal analysis unit 140 may analyze the physiological signal S and obtain at least one physiological signal parameter, such as the signal-to-noise ratio N, the root mean square value M, a resistance value, a waveform peak value or other physiological signal parameters. The strain signal analysis unit 140 may analyze the waveform of the physiological signal S to obtain the aforementioned physiological signal parameters by using any suitable mathematical method or analysis method. When the signal-to-noise ratio N, the root mean square value M, the resistance value and/or the waveform peak value are abnormal, the strain signal analysis unit 140 corrects the physiological signal S. In addition, the strain signal analysis unit 140 may also determine whether to correct the physiological signal S according to the resistance value between the human body 10 and the electrode 120. For example, the strain signal analysis unit 140 determines whether the resistance value is equal to a normal resistance value; when the resistance value is not equal to the normal resistance value (i.e., abnormal), it indicates that the braid fabric 110 is loose, and the strain signal analysis unit 140 corrects the physiological signal S. The normal resistance value is, for example, within a resistance range. The resistance range may depend on the actual situation, which is not limited by the embodiments of the disclosure, and the resistance range may be obtained in advance through experiments or software simulations, and stored in the strain signal analysis unit 140 or in a memory (not illustrated) accessible to the strain signal analysis unit 140. In addition, when the braid fabric 110 is completely detached from the human body 10, the resistance value may not be measured. In another embodiment, the strain signal analysis unit 140 may determine whether the peak value of the waveform of the physiological signal S is equal to the normal peak value; when the peak value of the waveform of the physiological signal S is not equal to the normal peak value (i.e., abnormal), it indicates that the braid fabric 110 may be loose or be installed in the wrong position, and the strain signal analysis unit 140 corrects the physiological signal S. In an embodiment, the normal peak value ranges, for example, between 3 millivolts (mV) and 5 millivolts (mV). In summary, the strain signal analysis unit 140 may determine whether to perform correction of the physiological signal S according to at least one physiological signal parameter (for example, at least one of the signal-to-noise ratio N, the root mean square value M, the resistance value and the waveform peak value).

As illustrated in FIG. 6A, the less the radius of curvature r of the braid fabric 110 is, the tighter the tightness of the braid fabric 110 on the human body 10 is, and the higher the strain-sensing signal V is; the greater the radius of curvature r of the braid fabric 110 is, the looser the fabric 110 on the human body 10 is, and the less the strain-sensing signal V is. As illustrated in FIG. 6B, the tightness T between 3 and 5 (hereinafter referred to as a normal region T′) is the most comfortable tightness for the human body 10, and thus the strain signal analysis unit 140 considers it abnormal and performs correction of the physiological signal S when the signal-to-noise ratio N exceeds the normal region T′. As illustrated in FIG. 6C, the tightness T between 3 and 5 (hereinafter referred to as the normal region T′) is the most comfortable tightness for the human body 10, and thus the strain signal analysis unit 140 considers it abnormal and corrects the physiological signal S when the root mean square value M exceeds the normal region T′. In addition, the normal region T′ is not limited to the aforementioned numerical range. Different types of the wearable physiological signal detection devices may have different numerical ranges, and/or the wearable physiological signal detection devices with different specifications may have different numerical ranges.

In addition, the aforementioned correction method may be any suitable mathematical operation, such as multiplication. For example, the strain signal analysis unit 140 may perform a multiplication operation on the gain value G and the physiological signal S (for example, S′=S×G) and use the product value as the corrected physiological signal S′.

The braid fabric in each of the foregoing embodiments includes at least one strain-sensing fiber layer, wherein each strain-sensing fiber layer may be electrically connected to the strain signal analysis unit 140. Taking the braid fabric 110A in FIG. 1A as an example, its strain-sensing fiber layer 112A is electrically connected to the strain signal analysis unit 140 to transmit the strain-sensing signal V to the strain signal analysis unit 140. Taking the braid fabric 110B in FIG. 1B as an example, each strain-sensing fiber layer 112B is electrically connected to the strain signal analysis unit 140 and may transmit its respective strain-sensing signal V to the strain signal analysis unit 140. Taking the braid fabric 110C in FIG. 1C as an example, each strain-sensing fiber layer 112C is electrically connected to the strain signal analysis unit 140 and may transmit its own strain-sensing signal V to the strain signal analysis unit 140. Taking the braid fabric 110D in FIG. 1D as an example, the strain-sensing fiber layer 112D is electrically connected to the strain signal analysis unit 140 to transmit the strain-sensing signal V to the strain signal analysis unit 140. Taking the braid fabric 110E in FIG. 1E as an example, each strain-sensing fiber layer 112A is electrically connected to the strain signal analysis unit 140 and may transmit its respective strain-sensing signal V to the strain signal analysis unit 140. Taking the braid fabric 110F in FIG. 1F as an example, each strain-sensing fiber layer 112A is electrically connected to the strain signal analysis unit 140 and may transmit its respective strain-sensing signal V to the strain signal analysis unit 140. Taking the braid fabric 110G in FIG. 1G as an example, each strain-sensing fiber layer 112G is electrically connected to the strain signal analysis unit 140 and may transmit its respective strain-sensing signal V to the strain signal analysis unit 140. Taking the braid fabric 110H in FIG. 1H as an example, each strain-sensing fiber layer 112A and each strain-sensing fiber layer 112H are electrically connected to the strain signal analysis unit 140 and may transmit their respective strain-sensing signals V to strain signal analysis unit 140. Taking the braid fabric 110I in FIG. 1I as an example, the strain-sensing fiber layer 112I is electrically connected to the strain signal analysis unit 140 to transmit the strain-sensing signal V to the strain signal analysis unit 140.

Depending on the cross-sectional structure of a plurality of the strain-sensing fiber layers, the strain signal analysis unit 140 may obtain the gain value G in different ways, as further examples will be described below.

A plurality of strain-sensing fiber layers 112 are located on the same layer in the braid fabric 110. For example, the strain signal analysis unit 140 is further configured to: receive a plurality of the strain-sensing signals V sensed by the strain-sensing fiber layers 112; obtain an average value of these strain-sensing signals V; and obtain the gain value G according to the average value. For example, from Table 1 above, the gain value G corresponding to the average value (i.e., the column of the strain-sensing signal V) is obtained.

For example, if a plurality of the strain-sensing fiber layers 112 are respectively located at different heights (for example, the braid fabrics in FIG. 2B, 2C or 2E), the strain signal analysis unit 140 is further configured to: receive the signals sensed by the strain-sensing fiber layers 112, a plurality of the strain-sensing signals V; determine the weight w of each strain-sensing fiber layer 112; obtain these weighted strain-sensing signals V′ (for example, V′=V×w) according to each strain-sensing signal V and the corresponding weight w; obtain a plurality of the weighted gain values G′ according to the weighted strain-sensing signals V′; obtain the average value of these weighted gain values G′; and obtain the gain value G according to the average value. In an embodiment, the weighted gain value G′ corresponding to the weighted strain-sensing signal V′ (i.e., the column corresponding to the strain-sensing signal V in Table 1) may be obtained from the above Table 1, and the corresponding average value (that is, the gain value G corresponding to the field of the strain-sensing signal V in Table 1). In addition, the strain-sensing fiber layers 112 located at different height layers in the braid fabric 110 may correspond to different weights w. For example, the weight of the strain-sensing fiber layer 112 that is closer to the human body 10 is smaller, but this is not intended to limit the embodiment of the present disclosure.

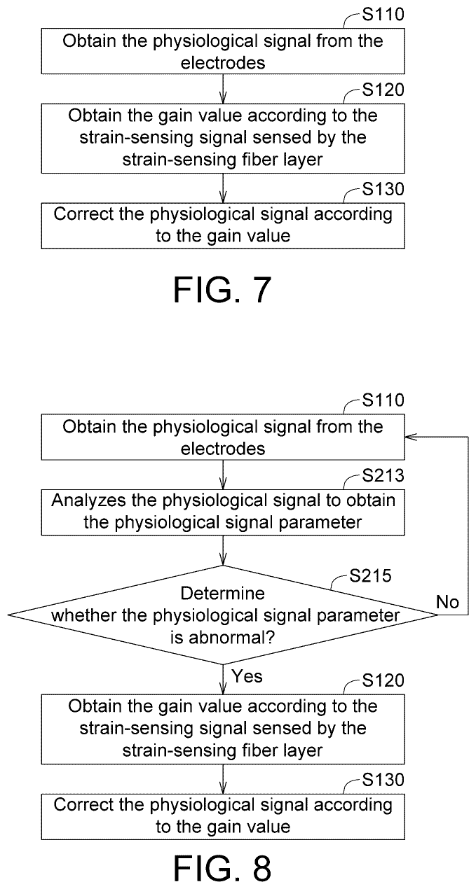

Referring to FIG. 7, FIG. 7 illustrates a flow chart of a physiological signal correction method of the wearable physiological signal detection device 100 in FIG. 5.

In step S110, the physiological signal analysis unit 130 obtains the physiological signal S from the electrodes 120. The physiological signal S is, for example, the electromyogram signal which is a fluctuating voltage. The waveform of the fluctuating voltage may depend on the tightness of the braid fabric 110 when the physiological signal detection device 100 is worn on the human body 10, the physiological state of the human body 10 or other factors.

In step S120, the strain signal analysis unit 140 obtains the gain value G according to the strain-sensing signal V sensed by the strain-sensing fiber layer 112. For example, the strain signal analysis unit 140 may obtain the gain value G corresponding to the strain-sensing signal V from Table 1 above.

In step S130, the strain signal analysis unit 140 corrects the physiological signal S according to the gain value G. For example, the strain signal analysis unit 140 may perform the multiplication operation on the gain value G and the physiological signal S and use the product value as the corrected physiological signal S′.

Referring to FIG. 8, FIG. 8 illustrates a flow chart of the physiological signal correction method of the wearable physiological signal detection device 100 in FIG. 5 according to another embodiment.

In step S110, the physiological signal analysis unit 130 obtains the physiological signal S from the electrodes 120. The physiological signal S is, for example, the electromyogram signal which is a fluctuating voltage. The waveform of the fluctuating voltage may depend on the tightness of the braid fabric 110 when the physiological signal detection device 100 is worn on the human body 10, the physiological state of the human body 10 or other factors.

In step S213, the strain signal analysis unit 140 analyzes the physiological signal S to obtain the physiological signal parameter. The physiological signal parameter is, for example, at least one of the aforementioned signal-to-noise ratio N, root mean square value M, resistance value and waveform peak value.

In step S215, the strain signal analysis unit 140 determines whether the physiological signal parameter is abnormal. For example, when at least one of the aforementioned signal-to-noise ratio N, root mean square value M, resistance value and peak value is abnormal, the strain signal analysis unit 140 determines that the physiological signal parameter is abnormal. When the physiological signal parameter is abnormal, the process proceeds to step S120; when the physiological signal parameter is normal, the process returns to step S110 to continuously detect whether the latest physiological signal S is abnormal.

In step S120, the strain signal analysis unit 140 obtains the gain value G according to the strain-sensing signal V sensed by the strain-sensing fiber layer 112. For example, the strain signal analysis unit 140 may obtain the gain value G corresponding to the strain-sensing signal V from Table 1 above.

In step S130, the strain signal analysis unit 140 corrects the physiological signal S according to the gain value G. For example, the strain signal analysis unit 140 may perform the multiplication operation on the gain value G and the physiological signal S and use the product value as the corrected physiological signal S′.

In summary, according to the braid fabric of this embodiment, the wearable physiological signal detection device and the physiological signal correction method using the same, the wearable physiological signal detection device may obtain the correction value (for example, the gain value) according to the physiological signal, and automatically correct physiological signals according to the correction value. In an embodiment, the wearable physiological signal detection device may determine whether the physiological signal is abnormal. When the physiological signal is abnormal, the wearable physiological signal detection device automatically corrects the physiological signal according to the correction value. In addition, the elastic fiber layer and the strain-sensing fiber layer of the braid fabric may be woven together and ultimately form one integrated fabric structure. The strain-sensing fiber layer will immediately and continuously feedback the wearing deformation status and generate the strain-sensing signal for correcting physiological signals.

It will be apparent to those skilled in the art that various modifications and variations could be made to the disclosed embodiments. It is intended that the specifications and examples be considered as exemplary only, with a true scope of the disclosure being indicated by the following claims and their equivalents.

What is claimed is:

1. A braid fabric, comprising:
an elastic fiber layer; and
a strain-sensing fiber layer assembled together with the elastic fiber layer.

2. The braid fabric according to claim 1, further comprising:
a plurality of the strain-sensing fiber layers disposed separately from each other on the elastic fiber layer.

3. The braid fabric according to claim 2, further comprising:
a plurality of the elastic fiber layers;
wherein one of the strain-sensing fiber layers is disposed in a local portion of one of the elastic fiber layers.

4. A wearable physiological signal detection device, comprising:
a braid fabric, comprising:
an elastic fiber layer; and
a strain-sensing fiber layer assembled together with the elastic fiber layer;
a plurality of electrodes disposed on the braid fabric;
a physiological signal analysis unit electrically connected to the electrodes and configured to:
obtain a physiological signal from the electrodes; and
a strain signal analysis unit electrically connected to the physiological signal analysis unit and the strain-sensing fiber layer, and configured to:
obtain a gain value according to a strain-sensing signal sensed by the strain-sensing fiber layer; and
correct the physiological signal according to the gain value.

5. The wearable physiological signal detection device according to claim 4, wherein the braid fabric comprises a plurality of the strain-sensing fiber layers; the strain signal analysis unit is further configured to:
receive a plurality of the strain-sensing signals sensed by the strain-sensing fiber layers;
obtain an average value of the strain-sensing signals; and
obtain the gain value according to the average value.

6. The wearable physiological signal detection device according to claim 4, wherein the braid fabric comprises a plurality of the strain-sensing fiber layers, and the strain-sensing fiber layers are layers of different heights in the braid fabric; the strain signal analysis unit is further configured to:
receive a plurality of the strain-sensing signals sensed by the strain-sensing fiber layers;
determine a weight of each strain-sensing fiber layer;
obtain a plurality of weighted strain-sensing signals according to each of the strain-sensing signals and the corresponding weight;
obtain a plurality of weighted gain values according to the weighted strain-sensing signals;
obtain an average value of the weighted gain values; and
obtain the gain value according to the average value.

7. The wearable physiological signal detection device according to claim 4, wherein the strain signal analysis unit is further configured to:
analyze the physiological signal to obtain a physiological signal parameter;
determine whether the physiological signal parameter is abnormal; and
obtain the gain value according to the strain-sensing signal sensed by the strain-sensing fiber layer if the physiological signal parameter is abnormal.

8. A physiological signal correction method, comprising:
obtaining a physiological signal from a plurality of electrodes by a physiological signal analysis unit, wherein the physiological signal analysis unit is electrically connected to the electrodes;
obtaining a gain value according to a strain-sensing signal sensed by a strain-sensing fiber layer, by a strain signal analysis unit, wherein the strain signal analysis unit electrically connected to the physiological signal analysis unit and the strain-sensing fiber layer; and
correcting the physiological signal according to the gain value by the strain signal analysis unit.

9. The physiological signal correction method according to claim 8, further comprising:
receiving a plurality of the strain-sensing signals sensed by a plurality of the strain-sensing fiber layers by the strain signal analysis unit;
obtaining an average value of the strain-sensing signals by the strain signal analysis unit; and
obtaining the gain value according to the average value by the strain signal analysis unit.

10. The physiological signal correction method according to claim 8, further comprising:
receiving a plurality of the strain-sensing signals sensed by a plurality of the strain-sensing fiber layers by the strain signal analysis unit;
determining a weight of each strain-sensing fiber layer by the strain signal analysis unit;
obtaining a plurality of weighted strain-sensing signals by the strain signal analysis unit according to each of the strain-sensing signals and the corresponding weight;
obtaining a plurality of weighted gain values according to the weighted strain-sensing signals by the strain signal analysis unit;
obtaining an average value of the weighted gain values by the strain signal analysis unit; and
obtaining the gain value according to the average value by the strain signal analysis unit.

11. The physiological signal correction method according to claim 8, further comprising:
analyzing the physiological signal to obtain a physiological signal parameter by the strain signal analysis unit;
determining whether the physiological signal parameter is abnormal by the strain signal analysis unit; and
obtaining the gain value according to the strain-sensing signal sensed by the strain-sensing fiber layer by the strain signal analysis unit if the physiological signal parameter is abnormal.
